(19) United States
(12) Patent Application Publication
(10) Pub. No.: US 2023/0265280 A1
(43) Pub. Date: Aug. 24, 2023

(54) COMPOSITION, MANUFACTURE, AND METHOD FOR PRODUCING COMPOSITION

(71) Applicant: Taichi NEMOTO, Shizuoka (JP)

(72) Inventor: Taichi NEMOTO, Shizuoka (JP)

(73) Assignee: Ricoh Company, Ltd., Tokyo (JP)

(21) Appl. No.: 18/004,408

(22) PCT Filed: Jun. 24, 2021
(86) PCT No.: PCT/IB2021/055584

§ 371 Date: Jan. 5, 2023

(30) Foreign Application Priority Data
Jul. 31, 2020 (JP) 2020-130966

(51) Int. Cl. C08L 67/02; C08L 67/04

(52) U.S. Cl. CPC C08L 67/02; C08L 67/04; C08L 2201/08; C08L 2203/02

(57) ABSTRACT

A composition includes a crystalline polymer and a compound that denatures at a temperature equal to or lower than a melting point of the crystalline polymer. The composition is substantially free from a solvent.

### TECHNICAL FIELD

The present disclosure relates to a composition, a manufacture, and a method for producing a composition.

### BACKGROUND ART

Preparations obtained by encapsulating a physiologically active substance (e.g., a medicine) with a base material (e.g., a resin) have been widely used. The physiologically active substance has characteristics involving denaturation, such as deactivation upon application of heat. Therefore, it is desired that the physiologically active substance be mixed with the base material without application of heat.

Examples of a method for encapsulating the physiologically active substance with the base material include a method where the physiologically active substance dissolved in water is added to the base material dissolved in an organic solvent, to mix the physiologically active substance and the base material. As the organic solvent, ethyl acetate, methylene chloride, chloroform, dimethyl formamide, tetrahydrofuran, or hexafluoroisopropanol is often used. However, many of the organic solvents are toxic to living organisms (see NPL 1). Therefore, proposed is a method where supercritical carbon oxide is used instead of the organic solvent, to melt a polymer to be the base material, to mix the physiologically active substance with the base material (see PTL 1).

However, the proposed method can use only a limited variety of the base materials, and cannot be used as an alternative for the method using an organic solvent.

### CITATION LIST

### Patent Literature

[PTL 1]
Japanese Translation of PCT International Application Publication No. JP-T-2000-51110

### Non Patent Literature

[NPL 1]
Drug Delivery System 23-6 2008

### SUMMARY OF INVENTION

### Technical Problem

The present disclosure has an object to provide a composition in which a compound that denatures at a temperature lower than a melting point of a base material is homogeneously dispersed in the base material.

### Solution to Problem

According to one aspect of the present disclosure, a composition includes a crystalline polymer, and a compound that denatures at a temperature equal to or lower than a melting point of the crystalline polymer. The composition is substantially free from a solvent.

### Advantageous Effects of Invention

According to the present disclosure, it is possible to provide a composition in which a compound that denatures at a temperature lower than a melting point of a base material is homogeneously dispersed in the base material.

### BRIEF DESCRIPTION OF DRAWINGS

The accompanying drawings are intended to depict example embodiments of the present invention and should not be interpreted to limit the scope thereof. The accompanying drawings are not to be considered as drawn to scale unless explicitly noted. Also, identical or similar reference numerals designate identical or similar components throughout the several views.

FIG. 1 is a phase diagram depicting a state of a substance relative to temperature and pressure.

FIG. 2 is a phase diagram for defining a range of a compressive fluid.

FIG. 3 is a schematic view illustrating one example of a batch-type device used for production of a composition of the present disclosure.

### DESCRIPTION OF EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise.

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

(Composition)

The composition of the present disclosure includes a crystalline polymer, and a compound that denatures at a temperature equal to or lower than a melting point of the crystalline polymer. The composition may further include other components according to the necessity. The composition of the present disclosure is substantially free from a solvent.

A solid composition of the present disclosure includes a crystalline polymer, and a compound that denatures at a temperature equal to or lower than a melting point of the crystalline polymer. The solid composition may further include other components according to the necessity.

Japanese Translation of PCT International Application Publication No. JP-T-2000-511110 proposes a method for producing a mixture including a physiologically active substance mixed with an amorphous aliphatic polyester resin.

However, the resin used in this proposal is limited to only polycaprolactone (weight average molecular weight: 4,000) that is one type of an aliphatic polyester resin. The polycaprolactone typically has a melting point of from 60 degrees C. through 80 degrees C., and a glass transition temperature equal to or lower than room temperature. There is no teaching in the literature above that a resin having a melting point of 90 degrees C. or higher or a glass transition temperature of 50 degrees C. or higher can be dissolved with liquid carbon dioxide of 40 degrees C. or lower, as with the polycaprolactone.

When carbon dioxide is used instead of an organic solvent as described above, applicable aliphatic polyesters are very limited. Therefore, the target decomposition rate in vivo cannot be obtained, and pharmaceutical preparations cannot be designed. Particularly, crystalline aliphatic polyester having a melting point of 90 degrees C. or higher or a glass transition temperature of 50 degrees C. or higher, which is considered to have low solubility in liquid carbon dioxide, cannot be used.

In the present disclosure, the phrase “substantially free from an organic solvent” means that an amount of the organic solvent in the composition, as measured by the following measuring method, is equal to or less than the detection limit.

<Measuring Method of Amount of Organic Solvent in Composition>

To 1 part by mass of the composition, which is a measurement target, 2 parts by mass of 2-propanol is added. After dispersing the resultant mixture by ultrasonic waves for 30 minutes, the resultant is stored in a refrigerator (5 degrees C.) for 1 day or longer to extract the organic solvent in the composition.

The liquid supernatant of the stored dispersion liquid is analyzed by gas chromatography (GC-14A, available from Shimadzu Corporation) to quantify the organic solvent and the residual monomers in the composition, to thereby measure a concentration of the organic solvent. The measuring conditions are as follows.
Device: Shimadzu GC-14A
Column: CBP20-M 50-0.25
Detector: FID
Injection amount: from 1 microliter through 5 microliters
Carrier gas: He 2.5 kg/cm²
Hydrogen flow rate: 0.6 kg/cm²
Air flow rate: 0.5 kg/cm²
Chart speed: 5 mm/min
Sensitivity: Range101×Atten20
Column temperature: 40 degrees C.
Injection temperature: 150 degrees C.

Crystalline polymer>

The crystalline polymer is a polymer having a clear melting point as measured by differential scanning calorimetry (DSC). The clear melting point means having an endothermic peak area of 1 g/J or greater as measured by the DSC, where the endothermic peak area indicates fusion of the crystal.

The crystalline polymer is not particularly limited and may be appropriately selected depending on the intended purpose, as long as it is a polymer having crystallinity. The crystalline polymer is preferably an aliphatic polyester resin (aliphatic polyester).

<<Aliphatic Polyester Resin>>

The aliphatic polyester resin has attracted attentions as a polymer material that is environmental friendly and gives low environmental loads, as the aliphatic polyester resin is biodegraded by microorganisms (see Structure and physical properties of aliphatic polyester, Biodegradable Polymer 2001, Vol. 50, No. 6, pp. 374-377).

Examples of the aliphatic polyester resin include, but are not limited to, polylactic acid, polyglycolic acid, poly(3-hydroxybutyrate), poly(3-hydroxybutyrate-3-hydroxyhexanoate), poly(3-hydroxybutyrate-3-hydroxyvalerate), polycaprolactone, polybutylene succinate, and poly(butylene succinate-adipate). These may be used alone or in combination. Many of them are resins having biodegradability or being decomposed in vivo.

Of these, polylactic acid, which is a carbon neutral material and relative inexpensive, is preferable.

The melting point of the crystalline polymer is preferably 90 degrees C. or higher, more preferably from 120 degrees C. through 250 degrees C. In the case where the crystalline polymer is a resin having biodegradability or being decomposed in vivo, the melting point of 90 degrees C. or higher is preferable because decomposition can be suppressed under conditions where occurrences of decomposition are not preferable (e.g., storage conditions). The melting point of the crystalline polymer can be measured by the below-described DSC.

The glass transition temperature (Tg) of the crystalline polymer is preferably 50 degrees C. or higher and more preferably from 55 degrees C. through 100 degrees C. The crystalline polymer having the glass transition temperature of 50 degrees C. or higher is preferable, because decomposition can be suppressed under conditions where occurrences of decomposition are not preferable (e.g., storage conditions).

The glass transition temperature of the crystalline polymer can be measured by the below-described DSC.

<DSC>

In the present disclosure, the melting point and glass transition temperature (Tg) can be measured, for example, by means of a DSC system (differential scanning calorimeter) (“Q-200,” available from TA Instruments Japan Inc.). Specifically, the glass transition temperature of the sample is measured in the following manner.

First, a sample container formed of aluminum is charged with about 5.0 mg of a sample, the sample container is placed in a holder unit, and the holder unit is set in an electric furnace. Subsequently, the sample is heated from 40 degrees C. to 200 degrees C. at a heating rate of 10 degrees C./min in a nitrogen atmosphere (first heating). Thereafter, the sample is cooled from 200 degrees C. to −15 degrees C. at a cooling rate of 10 degrees C./min, followed by heating to 200 degrees C. at a heating rate of 10 degrees C./min (second heating), to thereby measure a DSC curve.

The weight average molecular weight of the crystalline polymer is adjusted depending on the intended purpose, but the weight average molecular weight thereof is preferably 5,000 or greater but 30,000 or less.

<Measurement of Molecular Weight of Crystalline Polymer>
The molecular weight of the crystalline polymer can be measured by gel permeation
chromatography (GPC) under the following conditions.
Device: GPC-8020 (available from Tosoh Corporation)
Columns: TSK G2000HXL and G4000HXL (available from Tosoh Corporation)
Temperature: 40 degrees C.
Solvent: chloroform
Flow rate: 1.0 mL/min

A sample (1 mL) having a concentration of 0.5% by mass is injected, and a number average molecular weight Mn and a weight average molecular weight Mw of the polymer can be calculated from a molecular weight distribution of the polymer as measured under the conditions above, using a molecular weight calibration curve prepared from monodisperse polystyrene standard samples.

<Compound>

The compound has characteristics that the compound denatures at a temperature equal to or lower than the melting point of the crystalline polymer.

In the present disclosure, being denatured, denaturing, or denaturation means that characteristics of the compound change.

That the characteristics of the compound change means, for example, the following. Taking a protein as an example, it means that tissue loses its unique functions to have quantitative and qualitative changes and causes structural changes without cleavage of bonds to lose bioactivities. The protein thermally denatures at a high temperature, whereby a primary structure of the protein hardly changes but a secondary or higher-order structure is broken down. When heating egg white which is typically a transparent liquid, for example, water molecules that lightly bond to the surroundings to form a hydrated state are vibrated to loosen highly bonded sites, and a hydrophobic site encapsulated therein is exposed to cause a change to a white solid.

As the denaturation, there are reversible denaturation and irreversible denaturation. In the present disclosure, the denaturation may be any of reversible denaturation and irreversible denaturation.

As long as the compound is a known compound, whether the compound is denatured can be judged by identifying components from which the crystalline polymer is removed with an organic solvent.

Since the denaturation may thermodynamically exhibit characteristics of phase transition, the presence of the denaturation can be judged by such analysis as differential scanning calorimetry (DSC) or thermogravimetry/differential thermal analysis (TG-DTA). Specifically, the presence of the denaturation of the compound in the composition can be confirmed by measuring, as a reference, the data of the known compound and the known compound after denaturation.

The denaturation temperature of the compound is not particularly limited as long as the denaturation temperature is equal to or lower than the melting point of the crystalline polymer, and may be appropriately selected depending on the intended purpose. The denaturation temperature is preferably 60 degrees C. or lower.

The compound is not particularly limited as long as the compound denatures at a temperature equal to or lower than the melting point of the crystalline polymer, and may be appropriately selected depending on the intended purpose. Examples of the compound include, but are not limited to, polypeptide. In the present disclosure, the polypeptide is defined as a polypeptide that is a dimer or more multimer.

Examples of the polypeptide include, but are not limited to, proteins, enzymes, and antibodies. Specific examples thereof include, but are not limited to, antibiotics, nutritional supplements, metabolic modifiers, therapeutic agents, pain killers, and bioactive agents. Examples of the nutritional supplements include, but are not limited to, vitamins and minerals. Examples of the metabolic modifiers include, but are not limited to, starch, proteins, hormones, and appetite suppressants. Examples of the bioactive agents include, but are not limited to, vaccines. These may be used alone or in combination.

Of these, the compound is preferably a compound that is dissolved in the below-described compressive fluid or in water.

The amount of the compound is not particularly limited and may be appropriately selected depending on the intended purpose. The proportion of the compound is preferably 0.1% by mass or greater. The proportion of the compound can be measured by the following method.

<Measuring Method of Proportion of Compound Contained>

The proportion of the compound contained can be calculated from proportions of materials loaded. In the case where the proportions of materials are unknown, for example, the following gas chromatography mass spectrometry (GCMS) is performed, and the components can be determined through comparison with a known compound used as a standard sample. If necessary, calculation can be performed in combination with area ratios of a NMR spectrum or any other analysis methods.

-Measurement by GCMS-
GCMS: QP2010 available from Shimadzu Corporation, auxiliary device Py3030D available from Frontier Laboratories Ltd.
Separation column: Ultra ALLOY UA5-30M-0.25F available from Frontier Laboratories Ltd.
Sample heating temperature: 300 degrees C.
Column oven temperature: from 50 degrees C. (retained for 1 minute) by heating at 15 degrees C./min through 320 degrees C. (6 minutes)
Ionization method: Electron Ionization (E.I) method
Detection mass range: from 25 through 700 (m/z)

Other Components>

The composition and solid composition of the present disclosure may include other components according to the necessity.

The other components are not particularly limited, as long as the components do not denature the compound, and may be appropriately selected depending on the intended purpose. Examples thereof include, but are not limited to, amorphous polylactic acid, and amorphous resins obtained by copolymerizing lactide and caprolactone or glycolide.

The amount of the other components is not particularly limited and may be appropriately selected depending on the intended purpose.

(Manufacture)

The manufacture of the present disclosure includes the composition of the present disclosure, and may further include other components according to the necessity.

The other components are not particularly limited as long as the other components are components that can be used for typical resin products, and may be appropriately selected depending on the intended purpose.

Examples of the manufacture include, but are not limited to, molded products, films, particles, sheets, and fibers.

<Molded Product>

The molded product is a product obtained by processing the composition of the present disclosure using a mold. The term “molded product” includes, not only a molded product as a single unit, but also a part formed of a molded product, such as a handle of a tray, and a product equipped with a molded product, such as a tray equipped with handles.

The processing method using a mold is not particularly limited, and any of the methods known in the art can be used as the processing method.

The processing conditions at the time of molding are appropriately determined based on, for example, a kind of the composition of the present disclosure and an apparatus.

<Particles>

Examples of a method for forming the composition of the present disclosure into particles include, but are not limited to, a method for pulverizing the composition of the present disclosure according to a method known in the art.

The average particle diameter of the particles is not particularly limited and may be appropriately selected depending on the intended purpose. The average particle diameter thereof is preferably 1 micrometer or greater but 50 micrometers or less.

<Sheet>

The sheet is the composition of the present disclosure formed into a thin film, and is a sheet having a thickness of 250 micrometers or greater.

The sheet can be produced by applying a conventional production method of a sheet known in the art to the composition of the present disclosure.

The production method of the sheet is not particularly limited and may be appropriately selected depending on the intended purpose. Examples thereof include, but are not limited to, the T-die method, inflation, and calendaring.

The processing conditions for processing the composition into the sheet are not particularly limited and may be appropriately determined based on, for example, a kind of the composition and an apparatus.

<Film>

The film is the composition of the present disclosure formed into a thin film, and is a film having a thickness of less than 250 micrometers.

The film can be produced by stretch molding the composition.

The stretch molding is not particularly limited, but uniaxial stretch molding, or simultaneous or sequential biaxial stretch molding (e.g., the tubular method and the tenter method) applied for stretch molding of general plastics can be employed.

The thickness of the stretched film is not particularly limited and may be appropriately selected depending on the intended purpose. The thickness thereof is preferably 5 micrometers or greater but less than 250 micrometers.

Secondary processing may be performed on the molded stretched film for the purpose of imparting, for example, chemical functions, electrical functions, magnetic functions, mechanical functions, friction/abrasion/lubricant functions, optical functions, thermal functions, and surface functions such as biocompatibility. Examples of the secondary processing include, but are not limited to, embossing, coating, bonding, printing, metalizing (e.g., plating), mechanical processing, and a surface treatment (e.g., an antistatic treatment, a corona discharge treatment, a plasma treatment, a photochromism treatment, physical vapor deposition, chemical vapor deposition, and coating).

The stretched film can be applied for various uses, such as commodities, wrapping materials, medicines, materials for electrical devices, housing for home appliances, and materials for automobiles.

<Fibers>

The composition of the present disclosure can be applied for fibers, such as monofilaments and multi-filaments. The term “fibers” includes, not only fibers alone, such as monofilaments, but also an intermediate product formed of fibers, such as woven fabrics and non-woven fabrics, and a product including a woven fabric or non-woven fabric, such as masks.

In case of the monofilaments, the fibers are produced by melt spinning, cooling, and stretching the composition of the present disclosure to form the composition into fibers according to any of the methods known in the art. Depending on use thereof, a coating layer may be formed on a monofilament according to any of the methods known in the art, and the coating layer may include an antibacterial agent, a colorant, etc. In the case of being formed into the non-woven fabric, examples of production methods thereof include, but are not limited to, a method including melt spinning, cooling, stretching, opening fibers, depositing, and heat treating according to any of the methods known in the art.

(Method for Producing Composition)

The method of the present disclosure for producing a composition includes dissolving a crystalline polymer and a compound that denatures at a temperature equal to or lower than a melting point of the crystalline polymer in the presence of a compressive fluid and homogeneously mixing at a temperature at which the compound does not denature.

It has been known that a resin is generally plasticized by a compressive fluid to decrease in melt viscosity (see “Latest applied technology of supercritical fluid,” NTS Inc.).

In a case where carbon dioxide is used as a compressive fluid, however, the resin that is plasticized to the state of a liquid is limited to, for example, a low-molecular-weight amorphous resin, or a low-molecular-weight crystalline resin having a glass transition temperature equal to or lower than room temperature. Particularly, a polymer having a weight average molecular weight of 5,000 or greater, having crystallinity, and having a melting point of 90 degrees C. or higher cannot be mixed with the compound that denatures at a temperature equal to or lower than the melting point of the crystalline polymer at a temperature at which the compound does not denature. In case where a protein is the compound that denatures at a temperature equal to or lower than the melting point of the crystalline polymer, for example, it has been known that the protein denatures at a temperature range of from 60 degrees C. to 80 degrees C. Therefore, the temperature at which the protein does not denature is intended to be 60 degrees C. or lower.

Accordingly, the present inventors have diligently studied a structure of a compressive fluid in order to use the compressive fluid for mixing a crystalline polymer and a compound that denatures at a temperature equal to or lower than a melting point of the crystalline polymer. As a result, the present inventors have found that a crystalline polymer and a compound that denatures at a temperature equal to or lower than a melting point of the crystalline polymer can be mixed using dimethyl ether.

Mixing of the crystalline polymer and the compound is performed in the presence of the compressive fluid at a temperature equal to or lower than the melting point of the crystalline polymer. When chicken egg yolk is used as the compound, for example, mixing is performed at 60 degrees C. or lower because the denaturation temperature of the chicken egg yolk is 65 degrees C.

The temperature equal to or lower than the melting point of the crystalline polymer is, for example, a temperature equal to or lower than the temperature at which the compound denatures. Specifically, the temperature equal to or lower than the melting point of the crystalline polymer is preferably 60 degrees C. or lower and more preferably 50 degrees C. or lower.

<<Compressive Fluid>>

As the compressive fluid, dimethyl ether is used. Use of dimethyl ether is preferable for the following reasons, for example. Specifically, dimethyl ether has a critical pressure of about 5.3 MPa and a critical temperature of about 127 degrees C. to be able to easily create a supercritical state, dimethyl ether is harmless to human bodies as evident by use thereof as food additives, and dimethyl ether has a low greenhouse effect.

Note that, a compressive fluid of dimethyl ether may be added. Examples of a material that can be used in the state of a compressive fluid include, but are not limited to, carbon monoxide, carbon dioxide, nitrous oxide, nitrogen, methane, ethane, propane, perfluoroethane, perfluoropropane, 2,3-dimethylbutane, and ethylene.

The pressure at the time of mixing may be appropriately selected depending on a kind of the compressive fluid for use. In case of dimethyl ether, for example, the pressure for dissolving the crystalline polymer in the present disclosure is preferably 2.65 MPa or greater because the pressure at the critical point is 5.3 MPa.

The upper limit of the pressure at the time of mixing is not particularly limited as long as the pressure is within the range of the pressure over which the device for use can endure, and may be appropriately selected depending on the intended purpose. The upper limit of the pressure is preferably 50 MPa or less.

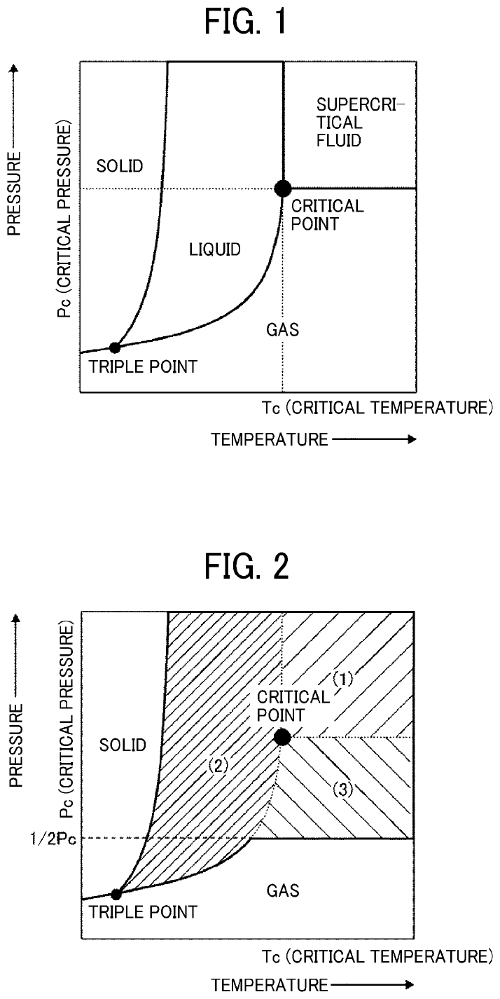

The compressive fluid used for the production of the composition will be described with reference to FIGS. 1 and 2. FIG. 1 is a phase diagram illustrating a state of a material relative to temperature and pressure. FIG. 2 is a phase diagram for defining a range of a compressive fluid. In the present embodiment, the term “compressive fluid” means a state of a material that is present in region (1), (2), or (3) of FIG. 2 in the phase diagram of FIG. 1.

It has been known that, in such a region, a material is in a state where the density thereof is extremely high, and behaves in a different manner from in the state of normal temperature and normal pressure. When the material is present in the region of (1), the material is in the state of a supercritical fluid. The supercritical fluid refers to a fluid that is present as a non-condensed high-density fluid in the temperature/pressure region exceeding the limit (critical point) where a gas and a liquid can co-exist, and the fluid is not condensed even when compressed. When the material is present in the region of (2), the material is in the state of a liquid, but the material is a liquidized gas obtained by compressing the material that is in the state of a gas at room temperature (25 degrees C.) and normal pressure (1 atm). When the material is present in the region of (3), the material is a high pressure gas having a pressure equal to or greater than ½ the critical pressure (Pc) (½ Pc).

Since the dissolution power of the compressive fluid varies depending on a combination of a resin species and a compressive fluid, a temperature, or a pressure, it is preferable that the supply amount of the compressive fluid be appropriately adjusted.

When the combination is a combination of polylactic acid and dimethyl ether, for example, the supply amount of dimethyl ether is preferably 50% by mass or greater. When the supply amount of dimethyl ether is 50% by mass or greater, a problem that polylactic acid cannot sufficiently dissolve can be prevented.

Examples of an apparatus usable for mixing the crystalline polymer and the compound include, but are not limited to, a batch-type polymerization reaction apparatus.

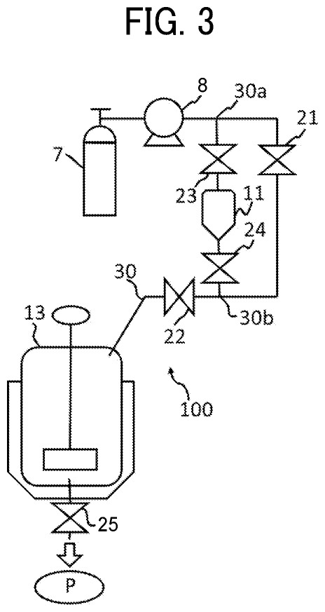

As one example of the batch-type polymerization reaction apparatus, a batch-type polymerization reaction apparatus 100 illustrated in FIG. 3 will be described. In the system diagram of FIG. 3, the polymerization reaction apparatus 100 includes a tank 7, a metering pump 8, an addition pot 11, a reaction vessel 13, and valves (21, 22, 23, 24, 25). Each device is coupled to a pressure resistant pipe 30 as illustrated in FIG. 3. The pipe 30 is provided with couplings (30a, 30b).

The tank 7 is configured to store a compressive fluid. The tank 7 may store gas or solid that is turned into a compressive fluid upon application of heat or pressure within a supply path leading to the reaction vessel 13 or within the reaction vessel 13. In this case, the gas or solid stored in the tank 7 is turned into the state of (1), (2), or (3) in the phase diagram of FIG. 2 within the reaction vessel 13 upon application of heat or pressure.

The metering pump 8 is configured to supply the compressive fluid stored in the tank 7 to the reaction vessel 13 at a constant pressure and flow rate. The addition pot 11 is configured to store a metal catalyst to be added to the raw materials inside the reaction vessel 13. The valves (21, 22, 23, 24) are each configured to be open or closed to switch the path of the compressive fluid stored in the tank 7, for example, between the path leading to the reaction vessel 13 via the addition pot 11 and the path leading the reaction vessel 13 without going through the addition pot 11.

The crystalline polymer before starting kneading is stored in the reaction vessel 13.

The crystalline polymer is melted or dissolved by either a method where the reaction vessel 13 is heated in advance to melt the crystalline polymer, followed by supplying a compressive fluid from the tank 7, or a method where the reaction vessel 13 is heated in the presence of a compressive fluid supplied from the tank 7. The resultant is cooled to a temperature at which a functional material unstable to heat is mixed. At this time, the crystalline polymer is in the state of a liquid.

In the case where the compound is not easily dissolved in the compressive fluid and is easily dissolved in water, the compound may be prepared as an aqueous solution in advance.

As a result, the reaction vessel 13 is capable of bringing the crystalline polymer in the state of liquid, stored in advance, the compressive fluid supplied from the tank 7, and the compound supplied from the addition pot 11 into contact with each other to mix them together. Note that, the reaction vessel 13 is a pressure resistant vessel, and may be equipped with a gas outlet from which evaporated substances are released.

As described above, the reaction vessel 13 includes a heater configured to heating raw materials and a compressive fluid, and a cooling function. The reaction vessel 13 further includes a stirring device configured to stir the crystalline polymer, the compound, and the compressive fluid. The valve 25 is opened after completion of a polymerization reaction to discharge the compressive fluid and the composition (polymer) from the reaction vessel 13.

### EXAMPLES

The present disclosure will be described below by way of Examples. The present disclosure should not be construed as being limited to these Examples. The term “part(s)” denotes “part(s) by mass,” unless otherwise specified.

### Example 1

A 100 mL reaction apparatus 100 illustrated in FIG. 3 was charged with 10 parts of polylactic acid (REVODE 110, obtained from HISUN, melting point: 160 degrees C., weight average molecular weight: 190,000) as the crystalline polymer, and the polylactic acid was heated to 150 degrees C., followed by adding 10 parts of dimethyl ether (abbreviated as “DME” in Table 1 below) as the compressive fluid so as to achieve an internal pressure of 5.5 MPa, to thereby melt the mixture. After the melting, the mixture was stirred in the reaction apparatus for 1 hour.

Subsequently, the resultant was cooled to 40 degrees C. Even when the internal temperature was decreased to 40 degrees C., the polylactic acid remained as a liquid, and the internal pressure indicated 0.5 MPa.

Next, to an addition pot 11 charged with 5 parts of water and 5 parts of chicken egg white (denaturation temperature: 71 degrees C.) as the compound that denatures at a temperature equal to or lower than a melting point of the crystalline polymer (hereinafter referred to as a “compound”), a compressive fluid (dimethyl ether) supplied from a tank 7 was supplied by opening a valve 23 to increase the pressure to a pressure of 1.0 MPa that was higher than the internal pressure (0.5 MPa) of the reaction apparatus 100, followed by closing the valve 23 and opening valves 24 and 22, to introduce the contents of the addition pot 11 to the reaction vessel 13 with the pressure.

The egg white was mixed with liquid polylactic acid. When the mixture became homogeneous, the valve 25 was opened to discharge the contents of the reaction vessel 13, to obtain a composition of Example 1.

### Example 2

A composition of Example 2 was obtained in the same manner as in Example 1, except that the crystalline polyester was replaced with polybutylene succinate (abbreviated as “PBS” in Table 1 below) (FZ71, obtained from Mitsubishi Chemical Corporation, weight average molecular weight: 210,000).

### Example 3

A composition of Example 3 was obtained in the same manner as in Example 1, except that the compound was replaced with chicken egg yolk (denaturation temperature: 65 degrees C.).

### Example 4

A composition of Example 4 was obtained in the same manner as in Example 1, except that the compressive fluid was replaced with a mixture (1:1) of perfluorobutane and dimethyl ether.

### Example 5

A composition of Example 5 was obtained in the same manner as in Example 1, except that the compressive fluid was replaced with a mixture (1:1) of carbon dioxide and dimethyl ether.

### Comparative Example 1

A composition of Comparative Example 1 was obtained in the same manner as in Example 1, except that the mixing temperature in the reaction apparatus was changed from 40 degrees C. to 100 degrees C.

Whether the solvent was included in the obtained composition was determined by the following method.

To 1 part by mass of the composition, 2 parts by mass of 2-propanol was added. The resultant was dispersed by ultrasonic waves for 30 minutes, followed by storing in a refrigerator (5 degrees C.) for 1 day or longer. After the storage, the organic solvent in the composition was extracted.

The liquid supernatant of the stored dispersion liquid was analyzed by gas chromatography (GC-14A, obtained from Shimadzu Corporation) to quantify the organic solvent and residual monomer in the composition, to measure an organic solvent concentration. The measuring conditions are as follows.
Device: Shimadzu GC-14A
Column: CBP20-M 50-0.25
Detector: FID
Injection amount: 1 microliter to 5 microliters
Carrier gas: He 2.5 kg/cm²
Hydrogen flow rate: 0.6 kg/cm²
Air flow rate: 0.5 kg/cm²
Chart speed: 5 mm/min
Sensitivity: Range 101×Atten 20
Column temperature: 40 degrees C.
Injection temperature: 150 degrees C.

As a result of the measurement above, no solvent was included in the compositions of Examples 1 to 5 and Comparative Example 1. Accordingly, it can be said that the compositions of Examples 1 to 5 and Comparative Example 1 are substantially free from the solvent.

Whether the compound in the composition was denatured was evaluated by the following method. Results are given in Table 1.

To 10 parts of the obtained composition, 90 parts of a solvent (chloroform) was added to dissolve the resin, and the compound was separated by filtration. The compound separated by filtration was added to water, and solubility thereof in water was observed.

The result where any difference had been observed in solubility was determined as “denatured,” and the result where the solubility had been similar was determined as “undenatured.”

TABLE 1

| | | | | | | | Evaluation |
|---|---|---|---|---|---|---|---|
| | Crystalline polymer | | Compound | | Production method | | result |
| | | Average molecular weight | | Amount | | Mixing | Denaturation |
| | Kind | (Mw) (×1,000) | Kind | (mass %) | Compressive fluid | temp. | (DSC) |
| Ex. 1 | polylactic acid | 20 | egg white | 50 | DME | 40 degrees C. | undenatured |
| Ex. 2 | PBS | 210 | egg white | 50 | DME | 40 degrees C. | undenatured |
| Ex. 3 | polylactic acid | 20 | egg yolk | 50 | DME | 40 degrees C. | undenatured |
| Ex. 4 | polylactic acid | 20 | egg white | 50 | perfluorobutane/DME | 40 degrees C. | undenatured |
| Ex. 5 | polylactic acid | 20 | egg white | 50 | carbon dioxide/DME | 40 degrees C. | undenatured |
| Comp. | polylactic acid | 20 | egg white | 50 | DME | 100 degrees C. | denatured |
| Ex. 1 | | | | | | | |

Embodiments of the present disclosure are, for example, as follows.
<1> A composition including:
a crystalline polymer; and
a compound that denatures at a temperature equal to or lower than a melting point of the crystalline polymer,
wherein the composition is substantially free from a solvent.
<2> A solid composition including:
a crystalline polymer; and
a compound that denatures at a temperature equal to or lower than a melting point of the crystalline polymer.
<3> The composition according to <1> or <2> above,
wherein the compound is at least one selected from the group consisting of proteins, enzymes, and antibodies.
<4> The composition according to any one of <1> to <3> above,
wherein the crystalline polymer has a melting point of 90 degrees C. or higher and a glass transition temperature of 50 degrees C. or higher.
<5> The composition according to any one of <1> to <4> above,
wherein the crystalline polymer has a weight average molecular weight of 5,000 or greater.
<6> The composition according to any one of <1> to <5> above,
wherein the crystalline polymer is an aliphatic polyester resin.
<7> The composition according to <6> above,
wherein the aliphatic polyester resin is at least one selected from the group consisting of polylactic acid, polybutylene succinate, and polyglycolic acid.
<8> The composition according to any one of <1> to <6> above,
wherein the compound that denatures at the temperature equal to or lower than the melting point of the crystalline polymer has a denaturation temperature of 60 degrees C. or lower.
<9> The composition according to any one of <1> to <8> above,
wherein a proportion of the compound that denatures at the temperature equal to or lower than the melting point of the crystalline polymer is 0.1% by mass or greater.
<10>A manufacture including
the composition according to any one of <1> to <9> above.
<11> The manufacture according to <10> above,
wherein the manufacture is at least one selected from the group consisting of molded products, films, particles, sheets, and fibers.
<12>A method for producing a composition, which includes a crystalline polymer, and a compound that denatures at a temperature equal to or lower than a melting point of the crystalline polymer, the method including mixing the crystalline polymer and the compound in presence of a compressive fluid.
<13> The method according to <12> above,
wherein the temperature equal to or lower than the melting point of the crystalline polymer is a temperature equal to or lower than a temperature at which the compound denatures.
<14> The method according to <12> or <13> above,
wherein the compressive fluid is at least one selected from the group consisting of dimethyl ether, perfluoroethane, and perfluoropropane.

The composition according to any one of <1> to <9> above, the manufacture according to <10> or <11> above, and the method for producing a composition according to any one of <12> to <14> above can solve the various problems existing in the art and can achieve the object of the present disclosure.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

This patent application is based on and claims priority to Japanese Patent Application No. 2020-130966, filed on Jul. 31, 2020, the entire disclosure of which is hereby incorporated by reference herein.

### REFERENCE SIGNS LIST

7 tank
8 metering pump
11 addition pot
13 reaction vessel
21, 22, 23, 24, 25 valves
30 pipe
30a, 30b couplings
100 polymerization reaction apparatus

1. A composition, comprising:
a crystalline polymer; and
a compound that denatures at a temperature equal to or lower than a melting point of the crystalline polymer,
wherein the composition is substantially free from a solvent.

2. A solid composition, comprising:
a crystalline polymer; and
a compound that denatures at a temperature equal to or lower than a melting point of the crystalline polymer.

3. The composition according to claim 1, wherein the compound is at least one selected from the group consisting of proteins, enzymes, and antibodies.

4. The composition according to claim 1, wherein the crystalline polymer has a melting point of 90 degrees C. or higher and a glass transition temperature of 50 degrees C. or higher.

5. The composition according to claim 1, wherein the crystalline polymer has a weight average molecular weight of 5,000 or greater.

6. The composition according to claim 1, wherein the crystalline polymer is an aliphatic polyester resin.

7. The composition according to claim 6, wherein the aliphatic polyester resin is at least one selected from the group consisting of polylactic acid, polybutylene succinate, and polyglycolic acid.

8. The composition according to claim 1, wherein the compound that denatures at the temperature equal to or lower than the melting point of the crystalline polymer has a denaturation temperature of 60 degrees C. or lower.

9. The composition according to claim 1, wherein a proportion of the compound that denatures at the temperature equal to or lower than the melting point of the crystalline polymer is 0.1% by mass or greater.

10. A manufacture, comprising:
the composition according to claim 1.

11. The manufacture according to claim 10, wherein the manufacture is at least one selected from the group consisting of molded products, films, particles, sheets, and fibers.

12. A method for producing a composition, which comprises a crystalline polymer, and a compound that denatures at a temperature equal to or lower than a melting point of the crystalline polymer, the method comprising;
mixing the crystalline polymer and the compound in presence of a compressive fluid.

13. The method according to claim 12, wherein the temperature equal to or lower than the melting point of the crystalline polymer is a temperature equal to or lower than a temperature at which the compound denatures.

14. The method according to claim 12, wherein the compressive fluid is at least one selected from the group consisting of dimethyl ether, perfluoroethane, and perfluoropropane.
